(19) United States
(12) Patent Application Publication
(10) Pub. No.: US 2014/0170727 A1
(43) Pub. Date: Jun. 19, 2014

(54) AFFINITY MEDIUM USING FIXED WHOLE CELLS

(71) Applicant: NANOMR, INC., Albuquerque, NM (US)

(72) Inventors: Sergey A. Dryga, Rio Rancho, NM (US); Robert Nadeau, Raymond, ME (US); Douglas Standridge, Albuquerque, NM (US)

(73) Assignee: NANOMR, INC., Albuquerque, NM (US)

(21) Appl. No.: 14/132,399

(22) Filed: Dec. 18, 2013

Related U.S. Application Data
(60) Provisional application No. 61/739,619, filed on Dec. 19, 2012.

(51) Int. Cl. C07K 16/12

(52) U.S. Cl. CPC C07K 16/1271
USPC 435/181; 435/268

(57) ABSTRACT

Separation media comprising a support and crosslinked whole cells fixed to the support. The separation media are useful in affinity columns for the separation of the antibodies from a solution, particularly for the separation of polyclonal antibodies that have been raised against the same type of whole cells. In an embodiment, the whole cells are whole bacterial cells.

### RELATED APPLICATION

The present application claims the benefit of and priority to U.S. provisional patent application Ser. No. 61/739,619 filed Dec. 19, 2012, the content of which is incorporated by reference herein in its entirety.

### FIELD OF THE INVENTION

The invention relates to separation media and methods for purifying antibodies that bind to antigens present on the surface of whole cells.

### BACKGROUND

Antibodies are “Y” shaped proteins that have antigen binding sites at the end of each arm of the “Y.” The antigen binding sites are very specific to an antigen structure, that is, there is essentially one unique antigen binding structure corresponding to each unique antigen, i.e., a “lock and key” mechanism. Thousands of antibodies are known and available from commercial suppliers. A comprehensive list of antibody suppliers is available from Linscott's directory (http://www.linscottsdirectory.com/search/antibodies).

Because of their exceptional specificity, antibodies are commonly used in biological detection techniques, such as Western blotting and ELISA. The antibodies can be directly labeled, e.g., with a fluorophore, heavy atom, or radionucleotide, or the antibodies can be modified to interact with other receptors or enzymes to result in an observable change after antibody binding. Using these labeling techniques, it is possible to quickly and accurately identify specific antigens of interest. The antigens may be, for example, markers of disease, such as a virus. In some systems, depending upon the labeling methods used, it is possible to detect markers at very low concentrations.

Antibodies can also be used to separate components of a biological sample. For example, as described in U.S. Patent Publication No. 2011/0262926, incorporated herein by reference, antibodies specific to a target may be coupled to a separation medium, e.g., a magnetic particle. When a sample is contacted to the separation medium, the antibodies bind the target molecules and the targets can then be removed by sequestering the separation medium, e.g., using a magnetic field. In a specific example, E. coli bacteria, present at just a few CFU/ml in blood, can be isolated from a sample.

Most antibodies used in detection and separation techniques are produced using monoclonal or polyclonal techniques. In monoclonal techniques a single cell line (hence monoclonal) is used to produce antibodies against a target substance (i.e., the antigen). Because all of the antibodies are from identical cells exposed to identical antigen, the resulting antibodies are substantially identical. Typically an animal (e.g., a mouse) is challenged with an antigen after which spleen cells from the animal are harvested and fused with a cell line (e.g., a myeloma cell line). The fused cells (hybridomas) are cultured and then assayed to identify the cells that are producing antibodies to the desired antigen. Once identified, the productive hybridomas are cultured, and become the single cell line capable of producing an unlimited supply of the desired antibodies. Monoclonal antibodies require careful preparation and culturing, take a fair amount of time to prepare, but can result in massive quantities of identical antibodies.

Polyclonal antibodies, in contrast, are raised by isolating antibodies directly from an antigen-challenged animal. That is, polyclonal antibodies are created via “normal” immunological pathways, which involve multiple B-cell variations naturally present within the animal. Because multiple B-cells are involved, the resultant antibodies, specific to the challenge antigen, have a number of different structures, corresponding to different epitopes (binding sites) on the antigen. For example, polyclonal antibodies raised against a virus are typically a mixture of antibodies that are specific to different sites on the surface of the virus. Because the antibodies are isolated directly from the serum of the animal, larger animals (e.g., goat, horse) may be used in order to increase the total volume of serum, and thus, the yield of antibodies. While monoclonal antibodies are uniform in performance (e.g., binding energy, specificity, etc.) polyclonal antibodies typically have a varied performance because they are a mixture of different antibodies for the same antigen.

Monoclonal antibody harvesting and purification typically involves lysing the hybridomas and removing cellular debris using filtration and/or centrifugation. After the debris is removed, the antibodies are purified using affinity chromatography whereby the valuable remnant is exposed to a column having a binding protein (i.e. A/G protein) or the antigen itself. After the antibody is bound to the column, the remnant can be washed away and the antibody later recovered from the column using an elution buffer. Because the monoclonal antibodies are substantially identical, the affinity column and elution buffer system can be “tuned” to recover the monoclonal antibodies with a high yield. That is, because all of the monoclonal antibodies are identical and are binding to the affinity column with the same binding characteristics, wash and elution buffers can be chosen to assure that all of the antibodies remain bound to the column during washing steps, and substantially all antibodies detach from the separation media during the elution step.

Harvesting and purifying polyclonal antibodies is typically not as facile because of the nature of the polyclonal antibodies. After the animal has been challenged (at least twice) an amount of blood is recovered from that animal, the serum from the blood is separated, e.g., using centrifugation. The valuable remnant may be exposed to an affinity column having a binding protein (i.e. A/G protein) or the antigen itself, i.e., as is done with monoclonal antibodies. However, the elution step is often much less reliable than in monoclonal antibodies. While an affinity column can be prepared using the antigen against which the polyclonal antibodies were raised or a similar structure, the elution efficiency will be variable because of the different binding energies of the different antibodies. Thus, when the serum background is washed away, some lower-binding-energy antibodies will also be washed from the column. During the elution step, some of the higher-binding-energy antibodies may remain bound to the column. Thus polyclonal antibody purification protocols often require multiple elution buffers (e.g., acetic acid and guanidinium chloride) to assure that all of the antibodies leave the separation media.

When whole cells are used as a challenge antigen in the production of polyclonal antibodies, affinity column separation is additionally complicated by the difficulty of duplicating the surface environment on the cell once it is attached to the support. For example, early research attempting to isolate cancer antibodies noted that whole cell-sepharose column chromatography was often ineffective at recovering antibodies from the serum of the challenge animal. In this example, the polyclonal antibodies were cultivated against a variety of compounds present on the cell surface of cancer cells under “normal” immunological conditions. However, when the cells were prepped to be affixed to sepharose, many of the surface compounds (e.g., glycoproteins) were modified such that they no longer corresponded to the proper antigens. Thus, efforts to purify the antibodies using immobilized whole cells were spotty, and did not result in purification of the broad array of antibodies that would be expected from polyclonal techniques. See Sela and Edelman “Isolation by Cell-Column Chromatography of Immunoglobulins Specific for Cell Surface Carbohydrates,” J. Exp. Med. 145, 443 (1977), incorporated herein by reference in its entirety. Attempts to purify polyclonal antibodies using cellular debris have been inconsistent for many of the same reasons.

Contemporary methods for isolating polyclonal antibodies raised against whole cells typically rely on collections of purified antigens, such as recombinant proteins, which are well-characterized, easily manipulated, and attachable to separation media. Of course, these methods still fail to recover the full array of antibodies that are produced by polyclonal methods, i.e., the antibodies that bind to antigens that are not among the collection of recombinant proteins.

In other instances, a serum containing the polyclonal antibodies is merely purified as far as possible, and the serum is used outright as a polyclonal antibody solution. This method allows the full range of polyclonal antibodies to be used, however, impurities from the serum may interfere with subsequent analytical techniques.

### SUMMARY

The disclosed invention solves many of the aforementioned problems by providing a separation medium that reliably binds most of the antibodies produced in a polyclonal process. The separation media includes fixed whole cells that are crosslinked, using aldehydes, for example, resulting in a robust network of proteins that are native to the whole cell surface. Because the whole cells are sequestered in a network of cross-linked proteins rather than prepped and chemically linked to an immobile phase, the whole cells maintain more of their in vivo surface characteristics and bind a wider variety of antibodies. Thus, upon elution from the fixed and crosslinked whole cell separation medium, a purified polyclonal antibody solution will have a wider variety of epitopes to the antigen against which they were raised, as well as a greater total amount of antibodies.

Using the methods of the invention, whole cells, such as whole cell bacteria, can be fixed and used as an affinity binding medium for the separation of antibodies from a solution. The fixed and crosslinked whole cells will typically be the same (or similar) to the whole cells against which the antibodies were initially raised and thus present the same panel of epitopes leading to overall greater retention of antibodies in the binding step. The whole cells may be fixed to any type of support, for example, bead or matrices. The support may be made of any compatible material, such as polymers or silica. In some embodiments, the whole cells may be inactivated whole cell bacteria, which is commercially-available for immunizations.

In one instance, the separation medium consists of a support, such as a bead, filament, or matrix, with crosslinked whole cells fixed to the support. In an embodiment, the whole cells are crosslinked with methylene bridges between surface proteins of the whole cells, using, for example, a formalin fixing solution. In some embodiments the whole cells are bacterial cells, for example, bacterial cells from the genera Listeria, Clostridium, Mycobacterium, Shigella, Borrelia, Campylobacter, Bacillus, Salmonella, Staphylococcus, Enterococcus, Pneumococcus, or Streptococcus. The separation media having the crosslinked whole cells fixed to the support may be used with an affinity column and for affinity separation of antibodies. Other useful scientific tools can be modified to be used as separation media using the techniques described herein. For example crosslinked whole cell bacteria can be fixed to a 96-well plate, and the wells used as a form of separation media for antibody preparation.

The disclosed separation media may be used to purify antibodies from a solution. In one method, a solution having antibodies is contacted with a separation medium comprising a support and crosslinked whole cells fixed to the support. After contacting the medium with a solution containing antibodies, the separation media with the antibodies bound thereto is washed, and then the antibodies are eluted to recover a purified antibody solution. The techniques may be used to purify both monoclonal and polyclonal antibodies, however the most benefit will be seen when purifying polyclonal antibodies that have been raised against the same (or a similar) whole cell. In one embodiment, the separation method is augmented by first exposing a solution containing the antibodies, e.g., animal serum, to a protein affinity column, e.g., protein G, which bind antibodies generally.

Fixed and crosslinked whole cell affinity media also avoid the pitfalls of using cellular debris as a separation medium. Whereas cellular debris affinity binding results in some amount of phantom binding due to interactions between antibodies and interior portions of the cell, as well as other debris, the whole cell media mostly interacts with properly binding antibodies, resulting in antibody preparations of a higher purity.

### DETAILED DESCRIPTION

The invention describes separation media comprising fixed and crosslinked whole cells, affinity columns comprising the separation media, and methods of using the separation media to purify antibodies from a solution. Using the separation media and methods described herein, it is possible to efficiently purify solutions of polyclonal antibodies. Because the separation media is a closer match to the original antigen, separation methods using the whole cell fixed media result in a greater portion of the antibodies being bound and ultimately recovered upon elution.

### Affinity Purification

Affinity purification generally involves incubating a sample containing target molecules (e.g., antibodies) with the affinity medium to allow the target molecules in the sample to bind to the immobilized target-binding species. The affinity medium with the target molecules is then washed to remove nonbound species from the medium. After the wash is complete, the affinity medium is rinsed with an elution buffer, causing the target molecules to dissociate from the medium. The elution wash, containing the target molecules, is recovered and may be neutralized or modified to make the solution more suitable for additional processing. Affinity purification of the target species can be quite efficient. For example, a single pass of a serum or cell-lysate sample through an affinity column can achieve greater than 1,000-fold purification of a specific protein, leaving only a single band after gel electrophoresis (e.g., SDS-PAGE) analysis.

Affinity columns with ligands that bind to general classes of proteins (e.g., antibodies) or commonly used fusion protein tags (e.g., 6xHis) are commercially available in pre-immobilized forms ready to use for affinity purification. Additionally it is known to use specialized linking chemistry to bind specific antibodies or antigens of interest. Most commonly, ligands are coupled directly to support media by formation of covalent chemical bonds between particular functional groups on the ligand (e.g., primary amines, sulfhydryls, carboxylic acids, aldehydes) and reactive groups on the support.

### Antibodies

It is well known that animals, particularly humans, undergo immunological changes when exposed to whole cells, such as bacteria. While the entire immunological mechanism is still not understood, it is clear that much of the change in immune response is due to development of antibodies in response to exposure to the whole cells, e.g., the antigens. It is also understood that a whole cell has a large number of surface features, e.g., proteins and glycoproteins, against which the body produces antibodies using a plethora of different B cells. The result is that a whole cell challenge results in hundreds or thousands of antibodies. These antibodies, in turn, become surface receptors for various cells involved in immunological pathways, and when the animal is again challenged with the antigen, the body produces lymphocytes, etc. to sequester and remove the antigens.

General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size, such as goats, dogs, sheep, mice, or camels may be immunized by administration of an amount of immunogen, such the target bacteria, effective to produce an immune response. An exemplary protocol is as follows. The animal is injected with 100 milligrams of antigen resuspended in adjuvant, for example Freund's complete adjuvant, dependent on the size of the animal, followed three weeks later with a subcutaneous injection of 100 micrograms to 100 milligrams of immunogen with adjuvant dependent on the size of the animal, for example Freund's incomplete adjuvant. Additional subcutaneous or intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity.

Techniques for producing monoclonal antibodies are known. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992, all of which are incorporated by reference herein in their entireties. These techniques can be used to produce antigen-reactive monoclonal antibodies, including antigen-specific IgG, and IgM monoclonal antibodies.

### Whole Cells

The techniques of the invention are generally applicable to whole cells that are useful for purifying antibodies. This includes whole animal cells, e.g., cancer cells, whole bacterial cells, or whole fungal cells. In certain instances, it will be necessary to raise and purify the cells to construct a separation medium comprising fixed and crosslinked whole cells. In other instances the cells are commercially available or available from a depository such as ATCC (Manassas, Va.).

Because there is a developed bacterial vaccine industry, whole bacterial cells are commercially available in a substantially purified form. Human vaccines against deadened whole bacteria are available for immunization against pertussis and tuberculosis, for example. Bacterial vaccinations, which are more common, are used to protect against diseases such as anthrax and bordetella (kennel cough). Accordingly, a variety of manufacturers, such as Organon Teknika, Corp. (a division of Merck; Raleigh, N.C.) and Pfizer Animal Health (New York, N.Y.) produce live and deadened bacteria for use in vaccines. These bacteria can be directly procured for use as antigens for the production of antibodies and also for preparation of fixed and crosslinked separation media.

Several types of bacterial whole cells may be used to prepare separation media of the invention. In certain embodiments, the bacterial whole cells comprise pathogenic bacteria. In other embodiments, the bacterial whole cells comprise gram positive or gram negative bacteria. Exemplary bacterial genera that may be used in separation media, affinity columns, and separation methods of the invention include E. coli, Listeria, Clostridium, Mycobacterium, Shigella, Borrelia, Campylobacter, Bacillus, Salmonella, Staphylococcus, Enterococcus, Pneumococcus, Streptococcus, and combinations thereof. Once constructed, separation media comprising bacterial whole cells may be used to isolate antibodies that have been raised against the bacteria. The antibodies may, in turn, be used for immunoassays (e.g., ELISA, Western Blot), for identifying pathogens in a clinical environment (e.g., medical or veterinary), or for detecting biological warfare agents.

### Cell Fixing

A variety of chemical cell-fixing methods are known for immobilizing whole cells to a surface, such as a microscope slide. Many of these techniques can also be used to construct an affinity medium for separating antibodies from a mixture. Using protocols for whole cell fixation on slides can be made generally applicable to fix whole cells on supports, thus producing a separation medium. Typically whole cell fixation methods that result in chemical crosslinking of proteins on the surface of the cells are favored, however, because these techniques result in a greater fidelity of cell surface structure. For example, aldehydes, such as formaldehyde and glutaraldehyde, that crosslink basic amino acids, can be used to produce an extended protein network that extends from cell to cell, and immobilizes the cells on the support. In some instances, alkylene bridges, e.g., comprising —(CH₂)ₙ— bonds, e.g., methlyene —CH₂— bonds, are formed between amino acid groups of proteins of nearby cells. When properly fixed and dried, the resulting web of crosslinked whole cells is robust, and can be stored under cool dry conditions for a period of weeks or months.

Using formalin (approximately 4% formaldehyde in phosphate buffered saline) fixing methods, for example, a whole cell sample can be mixed with an amount of support media and allowed to bind overnight at room temperature. Typically, the mixture of whole cells and support will undergo constant gentle agitation during the process to assure that the whole cells bind evenly on the support and adequately coat porous volumes within the support (if present). The formaldehyde in the formalin will cause crosslinking of amino acids on the surface of the cells, creating a networked coating on the surfaces of the support. After removing the formalin and rinsing the newly-created separation media, a polyclonal antibody solution that has been raised against the same whole cells can be introduced to the media. Because the whole cells retain most of their in vivo surface characteristics after formalin fixing, a wide variety of antibodies will bind to the separation media. After washing, the antibodies can be eluted as explained in more detail below.

### Supports

Useful affinity supports have high surface-area to volume ratios, minimal nonspecific binding properties, good flow characteristics, and mechanical and chemical stability. The underlying support material upon which the crosslinked whole cells are fixed may be varied depending upon the intended use of the separation media. The support may comprise small particles, such as beads, fibers, or filaments, or the support may have a regular structure such as a matrix or a frit. The beads, fibers, or filaments may have a dimension (e.g., a diameter) on the order of 10 to 200 μm, e.g., 30 to 150 μm, e.g., 50 to 100 μm. The support may have micro- and or macroscopic pores that provide greater surface area for whole cell binding while allowing hydrodynamic flow of the antibody solution as well as wash buffers and eluent. The pores may be on the order of less than 10 μm, e.g., less than 1 μm. The support may be made from materials such as silica, polymers (acrylamide, polypropylene, polystyrene, epoxides), and polysaccharides (agarose, cellulose, etc.) The support may also be a blend of materials, such as polymer beads with silanized surfaces. A commonly used support for antibody purification is crosslinked beaded agarose, which is typically available in 4% and 6% densities, i.e., 1 ml of saturated beads is more than 90% water by mass.

Typically, the support is constructed from a material that will not interfere with the binding and elution steps, and also will not react adversely to chemicals used during fixing, e.g., formaldehyde. A variety of commercially available supports are available for preparation as separation media, such as from BIORAD (Hercules, Calif.) or Life Technologies (Carlsbad, Calif.).

It is additionally possible that non-traditional support structures, e.g., well plates, microarrays, microfluidic systems, or computer chips may be prepared, e.g., with silanization methods, to receive fixed crosslinked whole cells to serve as a separation medium.

In one embodiment, isolated bacterial cells are mixed with silica beads in the presence of an excess of formalin, the mixture is agitated gently at room temperature for 24 hours, the formalin is poured off, the beads with the fixed bacterial cells are washed, and then allowed to dry. The prepared separation medium may be stored in cold (4° C.) to prolong shelf life.

### Columns

Columns, especially affinity columns, are flow tubes that hold a separation medium, and allow a solution containing target molecules to be introduced to the separation medium, bind to the separation medium, rinse away unbound species, and then release the target molecules while retaining the separation medium. Affinity columns come in a variety of diameters, depending upon the scale of the separation, and may be constructed to withstand positive and negative pressures. The interior diameter of the column may be less than 5 cm, e.g., less than 2 cm, e.g., less than 1 cm, e.g., less than 5 mm. The length may be at least 1 cm, e.g., at least 2 cm, e.g., at least 5 cm, e.g., at least 10 cm, e.g., at least 20 cm. The columns may include valves and fittings to control the flow of fluids into and out of the column. In many cases, the columns will have frits that will keep the separation medium from leaving the column as solutions are flowed through. The columns may be packed with techniques as simple as pouring a slurry of prepared separation media, e.g., media with crosslinked and fixed whole cells, into the column. Affinity columns are commercially available from a number of suppliers, including Life Technologies and Thermo-Fisher (Pittsburg, Pa.).

While separation media of the invention will most commonly be used with affinity columns, the separation media can be used with other containers, e.g., spin tubes, well plates, filters, and microfluidic systems.

### Binding

Typically, the conditions for binding antibodies to antigen-bound separation media should be close to the conditions that were originally experienced when the antibodies were raised against the specific (or similar) antigens. This assures that the binding mechanisms are substantially identical, and that only the antibodies that were produced in response to the antigen are retained on the separation media. Because antibodies are designed to recognize and bind antigens tightly under physiologic conditions, most affinity purification procedures use binding solutions that mimic physiologic pH and ionic strength. Typically a binding buffer is ionically balanced, such as phosphate-buffered saline (PBS) and/or Tris-buffered saline (TBS) at pH 7.2.

Once the antibody has been bound to the separation media, multiple washes of the same binding solution can be used to wash unbound material from the media. In some instances, additional salt or detergent can be added to the binding solution to disrupt any weak interactions. Such modifications can help to remove non-target materials that have non-specifically bound to the separation medium.

### Elution

Typically, the purified antibodies are eluted from the separation media by introducing elution solutions that have pH or ionic strength differences sufficient to disrupt the antigen-binding interaction. Most antibodies are moderately resilient, and can tolerate a range of pH from 2.5 to 11.5 without permanent inactivation. In some embodiments, low-pH solutions, i.e., pH=3.0 or lower are sufficient to completely dissociate the antibodies. Acidic elution solutions may comprise citric acid, HCl, or acidic amino acids, for example. In other embodiments, the elution solution may comprise triethylamine, phosphates, salts (e.g., LiCl, KCl, NaI, MgCl₂) or urea. In most instances, it will be necessary to return the eluted antibodies to physiological conditions, e.g., phosphate-buffered saline (PBS) and/or Tris-buffered saline (TBS) at pH 7.2, shortly after the antibodies are recovered from the media.

In some embodiments, multiple types of elution solutions may be used with the separation media described herein. A variety of antibody-antigen interactions will occur between the multitude of different antibodies in a polyclonal antibody solution and the multitude of different surface structures on the whole cells. In order be sure that all of the desired antibodies are removed from the support it may be necessary to contact the separation medium with multiple elution solutions of varying pH, ionic strength, etc. A variety of analytical techniques, e.g., UV/VIS/IR spectroscopy, fluorimetry, may be used to evaluate the concentration of antibodies in a given eluent sample in order to determine optimum elution conditions.

### EXAMPLES

### Example 1

### Isolation of Staphylococcus epidermidis Antibodies

A goat was immunized by first administering Staphylococcus epidermidis (ATCC) suspended in complete Freund's adjuvant intra lymph node, followed by subcutaneous injection of Staphylococcus epidermidis in incomplete Freund's adjuvant at 2 week intervals. The antigen (bacteria) was prepared for antibody production by growing the bacteria to exponential phase (OD600=0.4-0.8). Following harvest of the bacteria by centrifugation, the bacteria was inactivated using formalin fixation in 4% formaldehyde for 4 hr at 37° C. After 3 washes of bacteria with PBS (15 min wash, centrifugation for 20 min at 4000 rpm) the antigen concentration was measured using BCA assay and the antigen was used at 1 mg/mL for immunization.

After harvesting, the goat serum was first purified using affinity chromatography on a protein G sepharose column (GE Healthcare). Protein G is known to bind to the heavy chains of IgG antibodies, thus allowing a first clarification of the serum and removal of non-antibody proteins, cellular debris, nucleic acids, etc. A portion of the solution eluted from the column was evaluated for specific binding against Staphylococcus epidermidis using ELISA.

The remaining elution was then passed through an affinity column packed with porous silica beads that had been prepared as described above to fix whole Staphylococcus epidermidis cells to the surfaces of the beads. It was estimated that the molar ratio of whole cells antigens to antibody was 2:1. After binding, the column was washed with three washes of PBS buffer (pH 7.2) to remove additional debris as well as non-specifically binding antibodies. After washing, the Staphylococcus epidermidis antibodies were recovered using an elution solution containing guanidine and SDS. The resultant elution solution was neutralized and the antibody solution was evaluated for specific binding against Staphylococcus epidermidis using ELISA. The ELISA results showed that the subsequent affinity separation with crosslinked whole cell Staphylococcus epidermidis media resulted in a five-fold increase in specific binding as compared to the protein G affinity separation, alone. The yield of the second affinity separation was estimated at approximately 20% of the antibody content of the first (G protein) elution.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A support comprising whole cells that are crosslinked to the support.

2. The support of claim 1, wherein the whole cells are crosslinked with methylene bridges between surface proteins of the whole cells.

3. The support of claim 1, wherein the whole cells are fixed with formalin.

4. The support of claim 1, wherein the whole cells are bacterial cells.

5. The support of claim 4, wherein the bacterial cells are selected from the group consisting of E. coli, Listeria, Clostridium, Mycobacterium, Shigella, Borrelia, Campylobacter, Bacillus, Salmonella, Staphylococcus, Enterococcus, Pneumococcus, Streptococcus, and a combination thereof.

6. The support of claim 1, wherein the support comprises polymer beads or a polymer matrix.

7. The support of claim 1, wherein the support comprises silica beads or a silica matrix.

8. The support of claim 1, wherein the support is a well plate.

9. An affinity column comprising a support and crosslinked whole cells that are fixed to the support.

10. The affinity column of claim 9, wherein the whole cells are crosslinked with methylene bridges between surface proteins of the whole cells.

11. The affinity column of claim 9, wherein the whole cells are fixed with formalin.

12. The affinity column of claim 9, wherein the whole cells are bacterial cells.

13. The affinity column of claim 12, wherein the bacterial cells are selected from the group consisting of E. coli, Listeria, Clostridium, Mycobacterium, Shigella, Borrelia, Campylobacter, Bacillus, Salmonella, Staphylococcus, Enterococcus, Pneumococcus, Streptococcus, and a combination thereof.

14. The affinity column of claim 9, wherein the support comprises polymer beads or a polymer matrix.

15. The affinity column of claim 9, wherein the support comprises silica beads or a silica matrix.

16. A method for purifying antibodies from a solution, the method comprising:
contacting a solution comprising antibodies to a support comprising crosslinked whole cells that are fixed to the support under conditions such that at least one antibody binds the whole cells;
separating the bound antibodies from unbound antibodies; and
eluting the bound antibodies from the support.

17. The method of claim 16, wherein the whole cells are crosslinked with methylene bridges between surface proteins of the whole cells.

18. The method of claim 16, wherein the whole cells are fixed with formalin.

19. The method of claim 16, wherein the whole cells are bacterial cells.

20. The method of claim 19, wherein the bacterial cells are selected from the group consisting of E. coli, Listeria, Clostridium, Mycobacterium, Shigella, Borrelia, Campylobacter, Bacillus, Salmonella, Staphylococcus, Enterococcus, Pneumococcus, Streptococcus, and a combination thereof.

21. The method of claim 16, wherein the solution comprises monoclonal or polyclonal antibodies.

22. The method of claim 21, wherein the antibodies are from a mouse, a rabbit, or a goat.

23. The method of claim 16, wherein the molar ratio of antibodies to whole cell antigens is at least about 1:2.

24. The method of claim 16, further comprising:
contacting serum comprising the antibodies with a protein affinity column to bind the antibodies to the protein;
washing the protein affinity column with the antibodies bound thereto; and
eluting the antibodies from the protein affinity column, thereby creating a solution comprising antibodies.

25. The method of claim 24, wherein the protein is protein G.

26. The method of claim 16, wherein eluting the antibodies from the separation medium comprises contacting the antibodies bound to the separation medium with an elution buffer.
